(19) United States
(12) Patent Application Publication
(10) Pub. No.: US 2018/0256596 A1
(43) Pub. Date: Sep. 13, 2018

(54) METHOD AND TOPICAL COMPOSITION FOR THE TREATMENT OF ROSACEA AND SKIN ERYTHEMA USING PYRITHIONE ZINC

(71) Applicant: Paul Sutich, Long Branch, NJ (US)

(72) Inventor: Paul Sutich, Long Branch, NJ (US)

(21) Appl. No.: 15/976,346

(22) Filed: May 10, 2018

Related U.S. Application Data
(63) Continuation-in-part of application No. 15/450,239, filed on Mar. 6, 2017.

(51) Int. Cl. A61K 31/555; A61K 9/00; A61K 47/46; A61K 9/107

(52) U.S. Cl. CPC A61K 31/555; A61K 9/107; A61K 47/46; A61K 9/0014

(57) ABSTRACT

A method for the treatment of facial Rosacea and skin erythema using pyrithione zinc aqueous solution of given concentration strength as the sole active ingredient in a topically applied fluid composition in which the pyrithione zinc aqueous solution comprises about 20% to about 30% of the fluid composition by volume, with an inactive moisturized carrier comprising the remainder of the fluid composition by volume.

### CROSS-REFERENCE TO RELATED APPLICATIONS

This is a Continuation-in-Part of application Ser. No. 15/450,239 entitled Method And Topical Composition For The Treatment Of Rosacea And Skin Erythema Using Pyrithione Zinc filed Mar. 6, 2017, which is a Continuation-in-Part of application Ser. No. 14/594,735 filed Jan. 12, 2015.

### STATEMENT REGARDING FEDERALLY SPONSORED RESEARCH OR DEVELOPMENT

None.

### REFERENCE TO A MICROFICHE APPENDIX

None.

### BACKGROUND OF THE INVENTION

### Field of the Invention

This invention relates to the treatment of Rosacea and skin erythema, in general, and to a method utilizing a topical composition employing pyrithione zinc of different concentrations depending on the severity and extent of user symptoms, in particular.

### Description of the Related Art

Rosacea is a very common red, acne-like benign skin condition that affects approximately 45 million people worldwide. Its main symptoms include red or pink facial skin, small dilated blood vessels, small red bumps sometimes containing pus, cysts, and pink or irritated eyes. Characteristically involving the central region of the face (mainly the forehead, the cheeks, the chin, and the lower half of the nose), Rosacea is considered a chronic, long-term incurable skin condition with periodic ups and downs. Tending to occur more frequently in women, but more severely in men, Rosacea strikes both at potentially all ages. Unfortunately, emotional factors such as stress, fear, anxiety and embarrassment have been determined to aggravate the condition.

While lacking evidence that Rosacea can be spread by contact with the skin, the sharing of towels, of through inhalation, left untreated, the condition tends to worsen over time. Often being mistaken for acne, an allergic reaction, or other skin problems, its signs and symptoms may flare up for a period of weeks to months, and then diminish before flaring up again. Often aggravated by flushing, the redness in Rosacea may cause small blood vessels in the face to enlarge permanently and become more visible through the skin, appearing like tiny red lines. Continual or repeated episodes of flushing and blushing may promote inflammation, causing small red bumps often resembling teenage acne.

While not considered contagious or infectious—and even not necessarily requiring treatment if the individual is not bothered by the condition—many treatment choices are available depending on the severity and extent of symptoms. Antibacterial washes, topical medications, oral antibiotics, lasers, pulsed-light therapies, photodynamic therapy, and isotretinoin have oftentimes been utilized—with varying degrees of success and commensurate costs. Combination uses of them are not uncommon, with some being utilized alternatingly in the morning and at night, and others, such as with topical medications, more than once or twice a day. With metronidazole topical antibiotic medication (Rosadan®) and Azelaic Acid (Finacea®) costing in prescription amounts by dermatologists of $300.00 and more, it would be advantageous to have a simple pharmaceutical composition to effectively overcome the flushing and redness in the case of facial Rosacea, which is not yet achievable by the use of available treatments at a less expensive cost.

As will be understood, the same holds true for skin erythema, in general.

### Objects of the Present Invention

It is an object of the present invention, therefore, to provide an effective composition for the treatment of facial Rosacea.

It is an object of the present invention, also, to provide such a composition which is both easy to apply, and which can be obtained at a more affordable price.

It is an additional object of the invention to provide such a composition which for mild instances of facial Rosacea may be obtained over-the-counter, without the need for prescription.

It is a further object of the invention to provide a composition of greater concentration strength for more resistant and virulent conditions of facial Rosacea, obtainable by prescription.

It is another object of the present invention to provide such a composition for treating other conditions of the skin characterized by redness (i.e. skin erythema), and either as a gel, cream or lotion.

And, it is yet a further object of the invention to provide a common treatment of rosacea for increasingly resistant and virulent strains as well as for less serious rosacea-erythemas.

### SUMMARY OF THE INVENTION

As will become clear from the following description, the objects of the present invention are achieved through the employment of zinc pyrithione in the method of treatment. As will be understood by those skilled in the art, available in white-to-slightly yellow crystals and commercially supplied as a 24-26% aqueous solution, zinc pyrithione is also known by the following chemical names Bis [1-hydroxy-2(1H)-pyridinethionato-O,S] (T-4) zinc (IUPAC), pyrithione zinc, Zinc bis(2-pyridylthio)-N-oxide), Zinc pyridinethione, Zinc 2-pyridinethione-1-oxide, Bis (N-oxopyridine-2-thionato) zinc (II), ZP, ZnPT, ZnPTO, BOTZ; and under the tradenames Zinc Omadine and Vancide ZP—and also having the empirical formula C₁₀H₈N₂O₂S₂Zₙ. In a tested embodiment of the invention, the zinc pyrithione is combined as an aqueous solution with a lotion or cream cocoa butter moisturizer or spray carrier in forming a fluid composition for the treatment of the facial Rosacea. In accordance with the invention, its benefits follow with only pyrithione zinc employed as the active ingredient in the fluid composition—as contrasted with other proposed treatment compositions utilizing combinations of active ingredients interacting together.

### DETAILED DESCRIPTION OF THE INVENTION

Zinc pyrithione is currently regulated as a preservative in rinse-off products (excluding oral hygiene products) in strengths of up to 0.5% in general, and up to 1.0% in hair products. Furthermore, zinc pyrithione is also allowed in a strength up to 0.1% in leave-on hair products. More recently, it has been approved for use in rinse-off anti-dandruff hair care products in strengths from 1.0% to 2.0%. As such, zinc pyrithione (or pyrithione zinc) is commonly employed in zinc bar soaps or shampoos for treating and preventing itching, flaking and scaling of the scalp caused by dandruff or seborrhea, and available without prescription. In such bar soaps and shampoos (ZNP Bar®, DermaZinc Soap®, Head and Shoulders®), it is used with warm water to be massaged as a lather into the hair and scalp, being typically used once per day, or at least two times per week as directed by the user's physician.

In accordance with the present invention, and through clinical type observations, it has been determined that a topical application including pyrithione zinc aqueous solution of 1% concentration strength—(i.e. pyrithione zinc crystals of 1.0% concentration strength in a solution in which the solvent is water)—as the sole active ingredient could be effective when applied to the facial skin in significantly reducing the redness, flushing and inflammation associated with the chronic, incurable adult acne-like skin condition of facial Rosacea—and with skin erythema, in general.

A fluid composition of the invention may comprise all pharmaceutical forms for fluid administration including solutions, gels, lotions and creams—as well as ointments, foams, emulsions, micro-emulsions, milks, serums, aerosols, sprays, dispersions, micro-capsules and micro-particles thereof. Acceptable inactive carriers for the pyrithione zinc aqueous solution are those suitable for topical applications for the skin, will not cause any safety or toxicity concerns, and will be compatible with the pyrithione zinc aqueous solution. One such inactive carrier employed successfully with the pyrithione zinc aqueous solution has been found to be cocoa butter, an edible vegetable fat extracted from the cocoa bean. With the 1% concentration strength pyrthione zinc aqueous solution forming about 20% to about 30% of a moisturized fluid composition by volume, a cocoa butter emulsion carrier of about 3 parts cocoa butter to 1 part pyrithione zinc aqueous solution by volume (i.e. about 70% to about 80% inactive carrier in the fluid composition by volume) provided quite significant benefits of effectiveness to decrease the erythema resulting from facial Rosacea. Also utilizable as soaps and cleansing bars, a 1% concentration strength of pyrithione zinc aqueous solution has been approved by the FDA for cover-the-counter sales—and thus more readily obtainable, and at a lesser cost than other prescription medications used to treat these skin conditions.

Effective in most instances of facial Rosacea, more resistant and virulent situations and erythemas—and those where one user might not respond to a treatment as well as another user—such instances can be resolved by increasing the aqueous solution of pyrithione zinc from a 1% concentration strength to a 2%, 3% or 4% concentration strength by increasing the pyrithione zinc crystal content or concentration to the water solvent for the same 20%-30% volume relationship of the aqueous solution to the fluid composition; the 4% concentration strength being obtainable, however, only by way of dermatological prescription, for the fluid composition. Whether supplemented with known inactive bases such as excipients, binders, lubricants and disintegrants—or with selected oily materials or emulsifying agents—the treatment with this fluid composition of about 20% to about 30% pyrithione zinc aqueous solution by volume, with the inactive carriers being the remaining about 70% to about 80% volume, has been noted to be effective in a simple treatment application process. In fact, analysis has indicated that the fluid composition of the invention with its use of pyrithione zinc crystals in the aqueous solution can provide similar benefits for treating other skin complaints, conditions and afflictions—as with antibacterial agents, anti-acne agents, antiparasitic agents, antifungal agents, anti-inflammatory agents, and others. As such, with the pyrithione zinc aqueous solution, such other discreet erythema as acne and sunburn can be effectively reduced as well.

In this manner, there are four aspects of the invention: first, with a specific concentration strength, and of some 20%-30% by volume of pyrithione zinc aqueous solution in the fluid composition, an effective treatment of facial Rosacea could be had with only pyrithione zinc aqueous solution (that is—the pyrithione zinc crystals in the water solvent) needed as the sole active ingredient for the treatment, (thus reducing cost of manufacture of alternative treatments combining several active ingredients together of predetermined balanced percentages of each); second, more resistant conditions of facial Rosacea to treatment can be dealt with by increasing the concentration strength of the 20%-30% by volume pyrithione zinc aqueous solution utilized in the fluid composition; third, with the percentage of pyrithione zinc aqueous solution in the fluid composition of about 20% to about 30% pyrithione zinc aqueous solution by volume, increasing the concentration strength of the pyrithione zinc aqueous solution from a 1% concentration strength at the low end to a 4% concentration strength at the high end, substantially all resistance levels of facial Rosacea could be effectively treated—the moisturized inactive carrier comprising the remaining 70%-80% of the fluid composition by volume; and fourth, the concentration strength of the pyrithione zinc aqueous solution is determined by the concentration of pyrithione zinc crystals admixed in a solution in which the solvent is water.

In usage, the fluid composition of the invention is to be administered topically by a vehicle or carrier medium compatible with application to the skin. A useful regimen would be to a) mix 1 part of a 1% concentration strength pyrithione zinc aqueous solution with 3 parts of a cocoa butter emulsion moisturizer cream carrier; b) apply the fluid composition to the facial area of concern; c) leave the fluid composition for 2-3 minutes at the area of application; and d) rinse it off. The fluid composition will be seen to be highly effective in reducing the flushness and redness caused by facial Rosacea. For a more resistant condition of facial Rosacea, the regimen would be essentially the same—but with an increased concentration strength of pyrithione zinc aqueous solution, as by the addition of further pyrithione zinc (or zinc pyrithione) crystals to the aqueous formulation. In these instances, the benefits of the invention follow as long as the fluid composition treatment consists of about 20% to about 30% volume of pyrithione zinc aqueous solution with the 70%-80% moisturized carrier volume, without the need for an inclusion of any other active ingredient—thereby defining such volume percentage of pyrithione zinc crystal aqueous solution as the sole active ingredient of the fluid composition. In a preferred embodiment of the invention, the pyrithione zinc aqueous solution is employed as the sole active agent in the fluid composition—obtaining it by dispersing the white-to-yellow empirically defined pyrithione zinc in water to obtain the appropriate concentration strength, and of the 20%-30% aqueous solution volume amount.

Such benefits will thus be appreciated to include a more effective treatment, a treatment which requires less time for leaving the pharmaceutical form of composition on the face (such as 2-3 minutes), a decrease in the number of applications needed to treat a virulent Rosacea condition—and one which saves on the amount of product used for treating the condition no matter its extent, just by adjusting the crystals to achieve the 20%-30% relationship by volume of the desired concentration strength with the resulting 70%-80% relationship by volume with the inactive carrier component.

While there have been described what are considered to be preferred embodiments of the present invention, it will be readily appreciated by those skilled in the art that modifications can be made without departing from the scope of the teachings herein. For at least such reason, therefore, resort should be had to the claims appended hereto for a true understanding of the scope of the invention.

I claim:

1. A method of treating erythema resulting from facial Rosacea in a subject comprising the step of topically administering to the face of said subject a fluid composition consisting of an inactive moisturizer carrier and a single active ingredient, wherein the single active ingredient consists of a pyrithione zinc aqueous solution of about 20% to about 30% of the fluid composition by volume and the inactive moisturized carrier comprises the remainder of the fluid composition by volume, and wherein said pyrithione zinc aqueous solution included in the fluid composition is of a 1% to 4% concentration strength, and wherein said pyrithione zinc aqueous solution is the sole active ingredient of the fluid composition.

2. The method of claim 1 wherein a pharmaceutical form for administering the fluid composition includes an inactive moisturizer carrier selected from solutions, gels, lotions, creams, ointments, foams, emulsions, micro-emulsions, aerosols, sprays, and dispersions.

3. The method of claim 2 wherein a pharmaceutical form for administering said fluid composition includes about 20% to about 30% by volume of a pyrithione zinc aqueous solution of 1% to 4% concentration strength in a cocoa butter emulsion moisturizer cream carrier.

4. The method of claim 3 wherein said pharmaceutical form for administering said fluid composition consists of a mixture of one part pyrithione zinc aqueous solution by volume with 3 parts of a cocoa butter emulsion moisturizer cream carrier by volume.

5. A method of treating erythema resulting from facial Rosacea in a subject comprising the steps of: (a) topically administering to the facial area of said subject a fluid composition consisting of about 20% to about 30% of a pyrithione zinc aqueous solution of the fluid fluid composition by volume, wherein said 20% to about 30% of pyrithione zinc aqueous solution by volume is of a 1% to about 4% concentration strength, and is the sole active ingredient of the fluid composition; and (b) rinsing said fluid composition once administered from the face area of administration about 2-3 minutes thereafter.

6. The method of claim 5 wherein a pharmaceutical form for administering said fluid composition includes an inactive moisturizer carrier selected from solutions, gels, lotions, creams, ointments, foams, emulsions, micro-emulsions, aerosols sprays, and dispersions.

7. The method of claim 6 wherein a pharmaceutical form for administering said fluid composition in said treatment includes about 20% to about 30% by volume of a pyrithione zinc aqueous solution of 1% to 4% concentration strength in a cocoa butter emulsion moisturizer cream carrier.

8. The method of claim 7 wherein said pharmaceutical form for administering said fluid composition consists of a mixture of one part pyrithione zinc aqueous solution by volume
